(19) United States
(12) Patent Application Publication
(10) Pub. No.: US 2009/0042887 A1
(43) Pub. Date: Feb. 12, 2009

(54) Methods for Preventing or Reducing the Number of Gout Flares Using Xanthine Oxidoreductase Inhibitors and Anti-Inflammatory Agents

(75) Inventors: Christopher Lademacher, Chicago, IL (US); Patricia McDonald, Skokie (IL); Nancy Joseph Ridge, Highwood, IL (US); Rajneesh Taneja, Libertyville, IL (US)

Correspondence Address: DYKEMA GOSSETT PLLC, 10 S. WACKER DR., STE. 2300, CHICAGO, IL, 60606, US

(73) Assignee: TAP Pharmaceutical Products, Inc., Lake Forest, IL (US)

(21) Appl. No.: 12/015,527

(22) Filed: Jan. 17, 2008

Related U.S. Application Data
(60) Provisional application No. 60/881,794, filed on Jan. 19, 2007.

(51) Int. Cl. A61K 31/53; A61K 31/426; A61K 31/4439; A61P 29/00; A61K 31/415

(52) U.S. Cl. 514/246; 514/365; 514/406; 514/340; 514/338

(57) ABSTRACT

The present invention relates to methods of preventing gout flares in a subject in need thereof by administering to the subject a therapeutically effective amount of at least one xanthine oxidoreductase inhibiting compound or salt thereof and at least one non-steroidal anti-inflammatory drug for a period of six months on a regular basis.

### RELATED APPLICATION INFORMATION

This application claims priority to U.S. Application 60/881,794, filed on Jan. 19, 2007, the contents of which are herein incorporated by reference.

### FIELD OF THE INVENTION

The present invention relates to methods of treating subjects in order to prevent gout flares or reduce the number of gout flares for a period of at least six months in a subject afflicted with conditions such as hyperuricemia, gout, acute gouty arthritis, chronic gouty joint disease, tophaceous gout, uric acid nephropathy, and/or nephrolithiasis. More specifically, the present invention involves administering to a subject in need thereof of a therapeutically effective amount of at least one xanthine oxidoreductase inhibiting compound or salt thereof and at least one anti-inflammatory agent for a period of at least six (6) months.

### BACKGROUND OF THE INVENTION

Gout or gouty arthritis is one of the oldest known types of arthritis. Gout was identified first by the Egyptians in 2460 B.C. and then recognized by Hippocrates in the 5^{th} century B.C who referred to it as the “unwalkable disease”. Later, gout was known as the “Disease of Kings” due to its association with rich foods and alcohol consumption.

Today, gout is recognized as a disease characterized by hyperuricemia and recurrent episodes of acute joint inflammation that result from intra-articular deposition of urate as the monosodium salt in oversaturated tissue fluids. It is among the most common causes of acute monoarticular arthritis. In fact, estimates are that gout affects as many as 5 million Americans—twice the number of those affected with rheumatoid arthritis. While it is estimated that the overall incidence of gout among men and women is less than 1% (Pal, B., et al., Clin. Rheumatol., 19:21-25 (2000), Terkeltaub, R. A., N. Engl. J. Med., 349(17):1647-1655 (2003)), white males carry the major burden of this disease with a 8.6% cumulative incidence. (Roubenoff, R., et al., JAMA, 266:3004-3007 (1991)) In addition to gender, genetics also play a role in gout risk. Specifically, in the U.S., familial incidence of gout ranges from 6 to 18%. (Porter, R., Bull Hist. Med., 68:1-28 (1994)). Among hyperuricemic relatives of gout patients, the incidence of gout averages 20%. (Smyth, C. J., Metabolism, 6:218-229 (1957)).

A wide variety of theories exist to explain the increasing prevalence of gout. These include the rising tide of obesity (an estimated 60% of Americans are overweight), the aging population, the growing incidence of the metabolic syndrome and its components (for example hypertension, hyperlipidemia, impaired glucose tolerance), increased number of individuals with end-stage renal disease, and greater use of medications that may diminish uric acid excretion (for example diuretics and low-dose aspirin) (Bieber, J. D., Arthritis Rheum., 50(8):2400-2414 (2004), St-Onge, M. P., Am. J. Clin. Nutr., 78:1068-1073 (2003), Wallace, K. L., J. Rhematol., 31(8):1582-1587 (2004), Caspi, D., Arthritis Rheum., 43(1):103-108 (2000), Hajjar, I., JAMA, 200:199-206 (2003)). Among the elderly (e.g., those over age 65 years), a dramatic increase in the prevalence of gout has been recently reported, perhaps as a result of sustained hyperuricemia as well as other factors inherent to aging (for example, greater incidence of hypertension, use of medications that lower uric acid excretion, etc) (Wallace, K. L., J. Rhematol., 31(8):1582-1587 (2004)). Changes in dietary patterns have also been cited as factors that impact the incidence of gout. Recent epidemiologic data indicate that the increasing prevalence of gout is related to greater consumption of meats, seafood, and alcohol with beer posing a risk greater than that of liquor or wine. (Choi, H. K., et al., Lancet, 363(9417):1277-1281 (2004); Choi, H. K., N. Engl. J. Med., 350(11):1093-1103 (2004)).

As alluded to briefly above, gout is characterized by the symptomatic deposition of urate crystals in joint tissues as a result of urate supersaturation of extracellular fluids, a biochemical aberration reflected by hyperuricemia (serum urate levels exceeding 6.8). mg/dL. Initially, however, patients suffer from asymptomatic hyperuricemia, meaning that these patients have elevated serum urate levels in their blood for a period of time before having their first gout attack. An acute attack of gout is manifested by a highly inflammatory arthritis that is often accompanied by intense swelling, redness and warmth surrounding a joint caused by the movement of monosodium urate crystals in or out of the cell. In addition, chills, a low grade fever and an elevated white blood cell count can occur, mimicking an infection. These acute attacks of gout are also referred to as “gout flares”. After an initial attack, a patient may go for a period of months or years without or between gout attacks. After a number of years of gouty attacks, patients may develop a chronic arthritis that results in bone and cartilage destruction and deformity. Urate crystals deposit within and surrounding the joint thereby causing a chronic destructive inflammatory process.

Long-term restoration of serum urate levels to <6.0 mg/dL typically requires the use of an anti-hyperuricemic agent. Urate lowering therapy is recommended for subjects suffering from gout and one or more of the following conditions: acute gouty arthritis, chronic gouty joint disease, tophaceous gout, uric acid nephropathy and/or nephrolithiasis (kidney stones). However, subjects being treated with anti-hyperuricemic agents may also experience one or more acute gout attacks or gout flares after initiation of their treatment with anti-hyperuricemic agents. During an acute gout attack or gout flare, subjects typically receive additional therapy, such as one or more anti-inflammatory agents such as colchicine or a non-steroidal anti-inflammatory drug (“NSAID”). While many anti-inflammatory agents are known to be useful for treating acute gout attacks or flares, there is a need in the art to prevent these acute gout attacks or flares as well as to reduce the number of gout attacks or flares that a subject experiences during treatment to restore the subject's normal serum urate levels.

### SUMMARY OF THE PRESENT INVENTION

In one embodiment, the present invention relates to a method of preventing one or more gout flares in a subject, the method comprising the step of administering to the subject on a regular basis and for a period of at least six months, a therapeutically effective amount of at least one xanthine oxidoreductase inhibitor or a pharmaceutically acceptable salt thereof and a therapeutically effective amount of at least one anti-inflammatory agent.

In another embodiment, the present invention relates to a method of preventing one or more gout flares in a subject, the method comprising the step of administering to the subject on a regular basis and for a period of at least six months, a therapeutically effective amount of at least one anti-inflammatory agent and a therapeutically effective amount of a second compound or a pharmaceutically acceptable salt thereof, wherein said second compound comprises the formula:

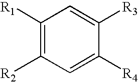

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

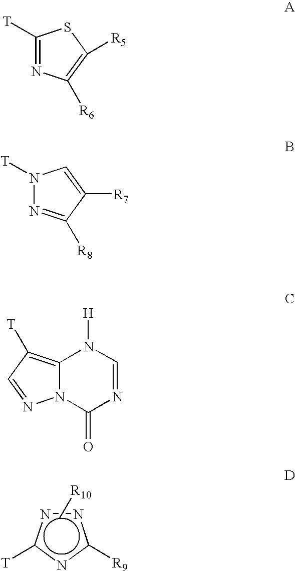

wherein T connects A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

In yet another embodiment, the present invention relates to a method of preventing one or more gout flares in a subject, the method comprising the step of administering to the subject on a regular basis and for a period of at least six months a therapeutically effective amount of at least one anti-inflammatory agent and a therapeutically effective amount of a second compound or a pharmaceutically acceptable salt thereof, wherein said second compound comprises the formula:

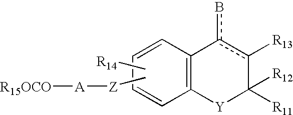

wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and the dotted line refers to either a single bond, a double bond, or two single bonds.

A subject being treated pursuant to the methods of the invention can have one or more of the following conditions: hyperuricemia, gout, acute gouty arthritis, chronic gouty joint disease, tophaceous gout, uric acid nephropathy, or nephrolithiasis.

The anti-inflammatory agent used in the above methods can be colchicine or one or more non-steroidal anti-inflammatory drugs (“NSAIDs”). NSAIDs used to treat subjects pursuant to the methods of the invention can be selected from the group consisting of: acetaminophen, amoxiprin, benorilate, choline magnesium salicylate, difunisal, faislamine, methyl salicylate, magnesium salicylate, salicyl salicylate, diclofenac, aceclofenac, acemetacin, bromfenac, etodolac, ketorolac, nabumetone, sulindac, tolmetin, ibuprofen, carprofen, fenbufen, fenoprofen, flurbiprofen, ketoprofen, loxoprofen, naproxen, tiaprofenic acid, mefenamic acid, meclofenamic acid, tolfenamic acid, phenylbutazone, azapropazone, metamizole, oxyphenbutazone, piroxicam, lornoxicam, meloxicam, tenoxicam, celecoxib, etoricoxib, lumiracoxib, parecoxib, nimesulide, licofelone, indomethacin, pharmaceutically acceptable salts thereof and mixtures thereof.

Additionally, the methods of the present invention can further comprise administering to the subject a therapeutically effective amount of at least one proton pump inhibitor (“PPI”). The PPI can be lansoprazole, ilaprazole, omeprazole, tenatoprazole, rabeprazole, esomeprazole, pantoprazole, pariprazole, leminoprazole or nepaprazole or a free base, a free acid, a salt, a hydrate, an ester, an amide, an enantiomer, an isomer, a tautomer, a polymorph, a prodrug or any derivative thereof.

The gout flares in a subject being treated pursuant to the methods of the present invention can be prevented for a period of time of at least six months, seven months, eight months, nine months, ten months, eleven months, twelve months, thirteen months, fourteen months, fifteen months, sixteen months, seventeen months, eighteen months, nineteen months, twenty months, twenty one months, twenty two months, twenty three months and twenty four months.

In yet another embodiment, the present invention relates to a pharmaceutical kit. The pharmaceutical kit of the present invention comprises as active ingredients a therapeutically effective amount of: (1) at least one xanthine oxidoreductase inhibitor; and (2) at least one anti-inflammatory agent. Optionally, the kit can also further comprise a therapeutically effective amount of at least one proton pump inhibitor (“PPI”). In the kit of the present invention, the at least one xanthine oxidoreductase inhibitor and the at least one anti-inflammatory agent can each be provided as separate, independent dosage forms (such as, but not limited to, at least two (2) dosage forms). Alternatively, the at least one xanthine oxidoreductase inhibitor and the at least one anti-inflammatory agent can be combined in a single, unified dosage form. In still another alternative, the at least one xanthine oxidoreductase inhibitor, the at least one anti-inflammatory agent and at least one PPI can each be provided as separate, independent dosage forms (such as, but not limited to, at least three (3) dosage forms). In yet still another alternative, the at least one xanthine oxidoreductase inhibitor, the at least one anti-inflammatory agent and at least one PPI can be combined in a single, unified dosage form. In yet still a further alternative, the at least one xanthine oxidoreductase inhibitor and at least one PPI can be combined in a single, unified dosage form and the at least one anti-inflammatory agent can be provided as a separate, independent dosage form. In still another alternative, the at least one anti-inflammatory agent the and at least one PPI can be combined in a single, unified dosage form and the at least one xanthine oxidoreductase inhibitor can be provided as a separate, independent dosage form.

The at least one anti-inflammatory agent used in the above kit can be colchicine or one or more non-steroidal anti-inflammatory drugs (“NSAIDs”). The NSAID used in the kit of the present invention can be selected from the group consisting of: acetaminophen, amoxiprin, benorilate, choline magnesium salicylate, difunisal, faislamine, methyl salicylate, magnesium salicylate, salicyl salicylate, diclofenac, aceclofenac, acemetacin, bromfenac, etodolac, ketorolac, nabumetone, sulindac, tolmetin, ibuprofen, carprofen, fenbufen, fenoprofen, flurbiprofen, ketoprofen, loxoprofen, naproxen, tiaprofenic acid, mefenamic acid, meclofenamic acid, tolfenamic acid, phenylbutazone, azapropazone, metamizole, oxyphenbutazone, piroxicam, lornoxicam, meloxicam, tenoxicam, celecoxib, etoricoxib, lumiracoxib, parecoxib, nimesulide, licofelone, indomethacin, a COX-2 inhibitor and pharmaceutically acceptable salts thereof and mixtures thereof. The PPI that can be used in the kit of the present invention can be lansoprazole, ilaprazole, omeprazole, tenatoprazole, rabeprazole, esomeprazole, pantoprazole, pariprazole, leminoprazole or nepaprazole or a free base, a free acid, a salt, a hydrate, an ester, an amide, an enantiomer, an isomer, a tautomer, a polymorph, a prodrug or any derivative thereof.

### BRIEF DESCRIPTION OF THE FIGURE

FIG. 1 provides a detailed schematic of the study described in Example 1.

### DETAILED DESCRIPTION OF THE INVENTION

### Definitions

The terms “administer”, “administering”, “administered” or “administration” refer to any manner of providing a drug (such as, a xanthine oxidoreductase inhibitor, an anti-inflammatory agent, a PPI or any combinations thereof) to a subject or patient. Routes of administration can be accomplished through any means known by those skilled in the art. Such means include, but are not limited to, oral, buccal, intravenous, subcutaneous, intramuscular, by inhalation and the like.

As used herein, the term “allopurinol” refers to 3,5,7,8-tetrazabicyclo[4.3.0]nona-3,5,9-trien-2-one.

As used herein, the term “anti-inflammatory agent(s)” refers to colchicine, one or more non-steroidal anti-inflammatory drugs (“NSAIDs”) or any combinations thereof.

As used herein, the term “pharmaceutically acceptable” includes moieties or compounds that are, within the scope of sound medical judgment, suitable for use in contact with the tissues of humans and lower animals without undue toxicity, irritation, allergic response, and the like, and are commensurate with a reasonable benefit/risk ratio.

As used herein, the term “subject” refers to an animal, preferably a mammal, including a human or non-human. The terms patient and subject may be used interchangeably herein.

The terms “therapeutically effective amount” or “prophylactically effective amount” of one or more drugs (namely, at least one xanthine oxidoreductase inhibitor or a salt thereof, at least one anti-inflammatory agent, at least one proton pump inhibitor or any combinations thereof) refers to a nontoxic but sufficient amount of one or more drugs to provide the desired effect of preventing gout flares or reducing the number of gout flares in a subject for at least six (6) months. In other words, these terms mean a sufficient amount of, for example, one or more pharmaceutical compositions containing at least one xanthine oxidoreductase inhibiting compound, at least one anti-inflammatory agent and optionally, at least one PPI, necessary to prevent gout flares or reduce the number of gout flares in a subject, at a reasonable benefit/risk ratio applicable to any medical treatment. As with other pharmaceuticals, it will be understood that the total daily usage of one or more pharmaceutical compositions of the present invention will be decided by a patient's attending physician within the scope of sound medical judgment. The specific therapeutically effective or prophylactically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the drug at levels lower than required to achieve the desired therapeutic effect and to gradually increase the dosage until the desired effect is achieved.

Accordingly, the amount of drug that is “effective” or “prophylactic” will vary from subject to subject, depending on the age and general condition of the individual, the particular drug or drugs, and the like. Thus, it is not always possible to specify an exact “therapeutically effective amount” or a “prophylactically effective amount”. However, an appropriate “therapeutically effective amount” or “prophylactically effective amount” in any individual case may be determined by one skilled in the art.

As used herein, the term “proton pump inhibitor” or “PPI” which are used interchangeable herein, refers to any acid labile active agents possessing pharmacological activity as an inhibitor of H⁺/K⁺-ATPase. A PPI may, if be desired, be in the form of a free base, free acid, salt, ester, hydrate, anhydrate, amide, enantiomer, isomer, tautomer, prodrug, polymorph, derivative or the like, provided that the free base, salt, ester, hydrate, amide, enantiomer, isomer, tautomer, prodrug or any other pharmacologically suitable derivative is therapeutically active or undergoes conversion within or outside the body to a therapeutically active form. Examples of PPIs that can be used in the present invention include, but are not limited to, lansoprazole, ilaprazole, omeprazole, tenatoprazole, rabeprazole, esomeprazole, pantoprazole, pariprazole, leminoprazole or nepaprazole or a free base, a free acid, a salt, a hydrate, an ester, an amide, an enantiomer, an isomer, a tautomer, a polymorph, a prodrug or any derivative thereof.

Proton pump inhibitors as well as their salts, hydrates, esters, amides, enantiomers, isomers, tautomers, polymorphs, prodrugs, and derivatives may be prepared using standard procedures known to those skilled in the art of synthetic organic chemistry. See, e.g., March, Advanced Organic Chemistry: Reactions, Mechanisms and Structure, 4th Ed. (New York: Wiley-Interscience, 1992); Leonard et al., Advanced Practical Organic Chemistry (1992); Howarth et al., Core Organic Chemistry (1998); and Weisermel et al., Industrial Organic Chemistry (2002).

“Pharmaceutically acceptable salts,” or “salts,” of a proton pump inhibitor include the salt of a proton pump inhibitor prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, B-hydroxybutyric, galactaric and galacturonic acids.

Acid addition salts of proton pump inhibitors can prepared from the free base forms using conventional methodology involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like.

An acid addition salt can be reconverted to the free base by treatment with a suitable base. Thereupon, also contemplated herein are acid addition salts of the proton pump inhibitors that are halide salt and which can be prepared using hydrochloric or hydrobromic acids. Additionally, the basic salts can be alkali metal salts, e.g., sodium salt.

Salt forms of proton pump inhibitors include, but are not limited to, a sodium salt form such as esomeprazole sodium, omeprazole sodium, rabeprazole sodium, pantoprazole sodium; or a magnesium salt form such as esomeprazole magnesium or omeprazole magnesium, described in U.S. Pat. No. 5,900,424; a calcium salt form; or a potassium salt form such as the potassium salt of esomeprazole, described in U.S. Pat. No. 6,511,996. Other salts of esomeprazole are described in U.S. Pat. Nos. 4,738,974 and 6,369,085. Salt forms of pantoprazole and lansoprazole are discussed in U.S. Pat. Nos. 4,758,579 and 4,628,098, respectively.

Preparation of Esters of Proton Pump Inhibitors Involves Functionalizing Hydroxyl and/or carboxyl groups that may be present within the molecular structure of the drug. Alternatively, the esters are acyl-substituted derivatives of free alcohol groups, e.g., moieties derived from carboxylic acids of the formula RCOOR₁ where R₁ is a lower alkyl group. Esters can be reconverted to the free acids, if desired, by using conventional procedures such as hydrogenolysis or hydrolysis.

“Amides” or proton pump inhibitors may be prepared using techniques known to those skilled in the art or described in the pertinent literature. For example, amides may be prepared from esters, using suitable amine reactants, or they may be prepared from an anhydride or an acid chloride by reaction with an amine group such as ammonia or a lower alkyl amine.

“Tautomers” of substituted bicyclic aryl-imidazoles include, e.g., tautomers of omeprazole such as those described in U.S. Pat. Nos. 6,262,085; 6,262,086; 6,268,385; 6,312,723; 6,316,020; 6,326,384; 6,369,087; and 6,444,689.

An example of an “isomer” of a substituted bicyclic aryl-imidazole is the isomer of omeprazole including but not limited to isomers described in: Oishi et al., Acta Cryst. (1989), C45, 1921-1923; U.S. Pat. No. 6,150,380; U.S. Patent Publication No. 02/0156284; and PCT Publication No. WO 02/085889.

Examples of “polymorphs” of proton pump inhibitors include, but are not limited to, those described in PCT Publication No. WO 92/08716, and U.S. Pat. Nos. 4,045,563; 4,182,766; 4,508,905; 4,628,098; 4,636,499; 4,689,333; 4,758,579; 4,783,974; 4,786,505; 4,808,596; 4,853,230; 5,026,560; 5,013,743; 5,035,899; 5,045,321; 5,045,552; 5,093,132; 5,093,342; 5,433,959; 5,464,632; 5,536,735; 5,576,025; 5,599,794; 5,629,305; 5,639,478; 5,690,960; 5,703,110; 5,705,517; 5,714,504; 5,731,006; 5,879,708; 5,900,424; 5,948,773; 5,997,903; 6,017,560; 6,123,962; 6,147,103; 6,150,380; 6,166,213; 6,191,148; 5,187,340; 6,268,385; 6,262,086; 6,262,085; 6,296,875; 6,316,020; 6,328,994; 6,326,384; 6,369,085; 6,369,087; 6,380,234; 6,428,810; 6,444,689; and 6,462,0577.

As used herein, the term “non-steroidal anti-inflammatory drug” or “NSAID” which are used interchangeable herein, refers to one or more active agents which when administered to a subject exhibit an analgesic effect, an antipyretic effect, an anti-inflammatory effect or any combinations of the aforementioned effects. Preferred NSAIDs for use in the methods of the present invention are: acetaminophen, amoxiprin, benorilate, choline magnesium salicylate, difunisal, faislamine, methyl salicylate, magnesium salicylate, salicyl salicylate, diclofenac, aceclofenac, acemetacin, bromfenac, etodolac, ketorolac, nabumetone, sulindac, tolmetin, ibuprofen, carprofen, fenbufen, fenoprofen, flurbiprofen, ketoprofen, loxoprofen, naproxen, tiaprofenic acid, mefenamic acid, meclofenamic acid, tolfenamic acid, phenylbutazone, azapropazone, metamizole, oxyphenbutazone, piroxicam, lomoxicam, meloxicam, tenoxicam, celecoxib, etoricoxib, lumiracoxib, parecoxib, nimesulide, licofelone, indomethacin, a COX-2 inhibitor or pharmaceutically acceptable salts thereof and mixtures thereof

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

As used herein, the term “xanthine oxidoreductase inhibitor” refers to any compound that (1) is an inhibitor of a xanthine oxidoreductase, such as, but not limited to, xanthine oxidase; and (2) chemically, does not contain a purine ring in its structure (i.e. is a “non-purine”). The phrase “xanthine oxidoreductase inhibitor” as defined herein also includes metabolites, polymorphs, solvates and prodrugs of the such compounds, including metabolites, polymorphs, solvates and prodrugs of the exemplary compounds described as Formula I and Formula II below. Examples of xanthine oxidoreductase inhibitors include, but are not limited to, 2-[4-(2-carboxypropoxy)-3-cyanophenyl]-4-methyl-5-thiazolecarboxylic acid and compounds having the following Formula I, Formula II or Formula III:

Compounds of Formula I:

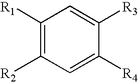

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

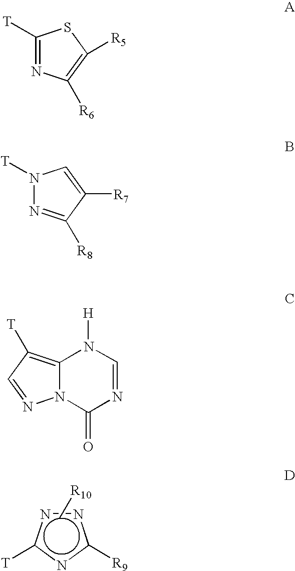

wherein T connects or attaches A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above in Formula I.

Compounds of Formula II:

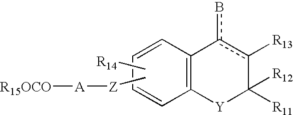

wherein R₁₁ and R₁₂ are each independently a hydrogen, a substituted or unsubstituted lower alkyl group, a substituted or unsubstituted phenyl (the substituted phenyl in this Formula II refers to a phenyl substituted with a halogen or lower alkyl, and the like. Examples include, but are not limited to, p-tolyl and p-chlorophenyl), or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl group, a substituted or unsubstituted phenyl (the substituted phenyl in this Formula II refers to a phenyl substituted with a halogen or lower alkyl group, and the like. Examples include, but are not limited to, p-tolyl and p-chlorophenyl), —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl group;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds (for example, when B is ethylenedithio, the dotted line shown in the ring structure can be two single bonds).

As used herein, the term “lower alkyl(s)” group refers to a C₁-C₇ alkyl group, including, but not limited to, including methyl, ethyl, n-propyl, isopropyl, butyl, isobutyl, sec-butyl, tert-butyl, pentyl, isopentyl, hexyl, heptal and the like.

As used herein, the term “lower alkoxy” refers to those groups formed by the bonding of a lower alkyl group to an oxygen atom, including, but not limited to, methoxy, ethoxy, propoxy, isopropoxy, butoxy, isobutoxy, pentoxy, hexoxy, heptoxy and the like.

As used herein, the term “lower alkylthio group” refers to those groups formed by the bonding of a lower alkyl to a sulfur atom.

As used herein, the term “halogen” refers to fluorine, chlorine, bromine and iodine.

As used herein, the term “substituted pyridyl” refers to a pyridyl group that can be substituted with a halogen, a cyano group, a lower alkyl, a lower alkoxy or a lower alkylthio group.

As used herein, the term “four- to eight-membered carbon ring” refers to cyclobutyl, cyclopentyl, cyclohexyl, cycloheptyl, cyclooctyl and the like.

As used herein, the phrase “pharmaceutically active ester-forming group” refers to a group which binds to a carboxyl group through an ester bond. Such ester-forming groups can be selected from carboxy-protecting groups commonly used for the preparation of pharmaceutically active substances, especially prodrugs. For the purpose of the invention, said group should be selected from those capable of binding to compounds having Formula II wherein R₁₅ is hydrogen through an ester bond. Resultant esters are effective to increase the stability, solubility, and absorption in gastrointestinal tract of the corresponding non-esterified forms of said compounds having Formula II, and also prolong the effective blood-level of it. Additionally, the ester bond can be cleaved easily at the pH of body fluid or by enzymatic actions in vivo to provide a biologically active form of the compound having Formula II. Preferred pharmaceutically active ester-forming groups include, but are not limited to, 1-(oxygen substituted)-C₂ to C₁₅ alkyl groups, for example, a straight, branched, ringed, or partially ringed alkanoyloxyalkyl groups, such as acetoxymethyl, acetoxyethyl, propionyloxymethyl, pivaloyloxymethyl, pivaloyloxyethyl, cyclohexaneacetoxyethyl, cyclohexanecarbonyloxycyclohexylmethyl, and the like, C₃ to C₁₅ alkoxycarbonyloxyalkyl groups, such as ethoxycarbonyloxyethyl, isopropoxycarbonyloxyethyl, isopropoxycarbonyloxypropyl, t-butoxycarbonyloxyethyl, isopentyloxycarbonyloxypropyl, cyclohexyloxycarbonyloxyethyl, cyclohexylmethoxycarbonyloxyethyl, bornyloxycarbonyloxyisopropyl, and the like, C₂ to C₈ alkoxyalkyls, such as methoxy methyl, methoxy ethyl, and the like, C₄ to C₈ 2-oxacycloalkyls such as, tetrahydropyranyl, tetrahydrofuranyl, and the like, substituted C₈ to C₁₂ aralkyls, for example, phenacyl, phthalidyl, and the like, C₆ to C₁₂ aryl, for example, phenyl xylyl, indanyl, and the like, C₂ to C₁₂ alkenyl, for example, allyl, (2-oxo-1,3-dioxolyl)methyl, and the like, and [4,5-dihydro-4-oxo-1H-pyrazolo[3,4-d]pyrimidin-1-yl]methyl, and the like.

In R₁₆ in Formula II, the term “ester” as used in the phrase “the ester of carboxymethyl” refers to a lower alkyl ester, such as methyl or ethyl ester; and the term “ether” used in the phrase “the ether of hydroxyethyl” means an ether which is formed by substitution of the hydrogen atom of hydroxyl group in the hydroxyethyl group by aliphatic or aromatic alkyl group, such as benzyl.

The carboxy-protecting groups may be substituted in various ways. Examples of substituents include halogen atom, alkyl groups, alkoxy groups, alkylthio groups and carboxy groups.

As used herein, the term “straight or branched hydrocarbon radical” in the definition of A in Formula II above refers to methylene, ethylene, propylene, methylmethylene, or isopropylene.

As used herein, the substituent of the “substituted nitrogen” in the definition of Y and Z in Formula II above are hydrogen, lower alkyl, or acyl.

As used herein, the term “phenyl-substituted lower alkyl” refers to a lower alkyl group substituted with phenyl, such as benzyl, phenethyl or phenylpropyl.

As used herein, the term “prodrug” refers to a derivative of the compounds shown in the above-described Formula I and Formula II that have chemically or metabolically cleavable groups and become by solvolysis or under physiological conditions compounds that are pharmaceutically active in vivo. Esters of carboxylic acids are an example of prodrugs that can be used in the dosage forms of the present invention. Methyl ester prodrugs may be prepared by reaction of a compound having the above-described formula in a medium such as methanol with an acid or base esterification catalyst (e.g., NaOH, H₂SO₄). Ethyl ester prodrugs are prepared in similar fashion using ethanol in place of methanol.

Compounds of Formula III:

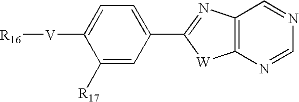

wherein R₁₆ is a phenyl or pyridyl each optionally having as a substituent, C₁-C₈ alkyl, C₁-C₈ haloalkyl, C₁-C₈ alkoxy, carboxy, halogen, hydroxy, nitro, cyano or an amino group;

wherein R₁₇ is a cyano or nitro group;

V is an oxygen or sulfur; and

W is a sulfur or NH.

Examples of compounds having the above Formula I are: 2-[3-cyano-4-(2-methylpropoxy)phenyl]-4-methylthiazole-5-carboxylic acid (also known as “febuxostat”), 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo[1,5-a]-1,3,5-triazin-4-(1H)-one, 8-[3-methoxy-4-(phenylsulfinyl)phenyl]-sodium salt (±) or 3-(2-methyl-4-pyridyl)-5-cyano-4-isobutoxyphenyl)-1,2,4-triazole.

Preferred compounds having the above Formula I are: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid. These preferred compounds have also been found not have an effect at a therapeutically effective amount in a subject on the activity of any of the following enzymes involved in purine and pyrimidine metabolism: guanine deaminase, hypoxanthine-guanine phosphoribosyltransferse, purine nucleotide phosphorylase, orotate phosphoribosyltransferase or orotidine-5-monophosphate decarboxylase (i.e., meaning that it is “selective” for none of these enzymes which are involved in purine and pyrimidine metabolism). Assays for determining the activity for each of the above-described enzymes is described in Yasuhiro Takano, et al., Life Sciences, 76:1835-1847 (2005). These preferred compounds have also been referred to in the literature as nonpurine, selective inhibitors of xathine oxidase (NP/SIXO).

Examples of compounds having the above Formula II are described in U.S. Pat. No. 5,268,386 and EP 0 415 566 A1.

Examples of compounds having the above Formula III are described in WO 2007/004688.

With the exception of pyrazolo[1,5-a]-1,3,5-triazin-4-(1H)-one, 8-[3-methoxy-4-(phenylsulfinyl)phenyl]-sodium salt (±), methods for making xanthine oxidoreductase inhibiting compounds of Formulas I and II for use in the methods of the present invention are known in the art and are described, for example, in U.S. Pat. Nos. 5,268,386, 5,614,520, 6,225,474, 7,074,816 and EP 0 415 566 A1 and in the publications Ishibuchi, S. et al., Bioorg. Med. Chem. Lett., 11:879-882 (2001) and which are each herein incorporated by reference. Other xanthine oxidoreductase inhibiting compounds can be found using xanthine oxidoreductase and xanthine in assays to determine if such candidate compounds inhibit conversion of xanthine into uric acid. Such assays are well known in the art.

Pyrazolo[1,5-a]-1,3,5-triazin-4-(1H)-one, 8-[3-methoxy-4-(phenylsulfinyl)phenyl]-sodium salt (±) is available from Otsuka Pharmaceutical Co. Ltd. (Tokyo, Japan) and is described in the following publications: Uematsu T., et al., “Pharmacokinetic and Pharmacodynamic Properties of a Novel Xanthine Oxidase Inhibitor, BOF-4272, in Healthy Volunteers, J. Pharmacology and Experimental Therapeutics, 270:453-459 (August 1994), Sato, S., A Novel Xanthine Deydrogenase Inhibitor (BOF-4272). In Purine and Pyrimidine Metabolism in Man, Vol. VII, Part A, ed. By P. A. Harkness, pp. 135-138, Plenum Press, New York. Pyrazolo[1,5-a]-1,3,5-triazin-4-(1H)-one, 8-[3-methoxy-4-(phenylsulfinyl)phenyl]-sodium salt (±) can be made using routine techniques known in the art.

### DETAILED DESCRIPTION OF THE PRESENT INVENTION

As mentioned briefly above, the present invention relates to methods of preventing gout flares or reducing the number of gout flares for a period of at least six (6) months in subjects in need thereof. Specifically, it has been discovered that the administration of a class of compounds known as xanthine oxidoreductase inhibitors with one or more anti-inflammatory agents on a regular basis for at least six (6) months prevents gout flares or reduces the number of gout flares experienced or suffered by a subject during said treatment period (namely, at least six (6) months). Subjects being treated with one or more xanthine oxidoreductase inhibitors may also experience one or more acute gout attacks or gout flares after initiation of their treatment with said inhibitors.

Because the xanthine oxidoreductase inhibitors of the present invention are effective in reducing serum urate levels, these compounds can be used to treat subjects suffering from hyperuricemia, gout, acute gouty arthritis, chronic gouty disease, tophaceous gout, uric acid nephropathy, and/or nephrolithiasis. Such treatments involve the administration of sufficient amounts of at least one xanthine oxidoreductase inhibitor to reduce a subject's serum urate level to <6.0 mg/dL for a prolonged period, preferably for at least six months, more preferably for at least a year, still more preferably for at least two years, and still more preferably for in excess of 30 months and beyond.

It has been discovered that the administration of at least one xanthine oxidoreductase inhibitor and at least one anti-inflammatory agents for a period of at least six (6) months to a subject suffering from hyperuricemia, gout, acute gouty arthritis, chronic gouty disease, tophaceous gout, uric acid nephropathy, and/or nephrolithiasis is useful for preventing gout flares or reducing the number of gout flares experienced by the subject during the six (6) month treatment period. The present invention also contemplates that the methods described herein can also be used to prevent gout flares or reduce the number of gout flares in a subject who is being treated pursuant to the methods described herein for a period of time longer than six (6) months, namely, a period of time of at least seven months, eight months, nine months, ten months, eleven months, twelve months, thirteen months, fourteen months, fifteen months, sixteen months, seventeen months, eighteen months, nineteen months, twenty months, twenty one months, twenty two months, twenty three months or twenty four months.

In one aspect, the one or more xanthine oxidoreductase inhibitors and one or more anti-inflammatory agents used in the methods of the present invention can be delivered and administered to a subject as separate, independent formulations or dosage forms (for example, but not limited to, two or more tablets or capsules, such as a first tablet or capsule containing one or more xanthine oxidoreductase inhibitors and a second tablet or capsule containing one or more anti-inflammatory agents (such as one or more NSAIDs)). If the one or more xanthine oxidoreductase inhibitors and one or more anti-inflammatory agents are to be administered to the subject as separate, independent pharmaceutical formulations, then the formulations can be administered (or dosed) to the subject sequentially, meaning that two or more formulations are administered to the subject immediately one right after another on the same day. Alternatively, the formulations can be administered to the subject intermittently on the same day or on different days. For example, one or more tablets or capsules containing one or more anti-inflammatory agents can be administered to a subject at some point during a day (such as in the morning before or after breakfast) and one or more tablets or capsules containing one or more xanthine oxidoreductase inhibitors can be administered (dosed) to the same subject 5 minutes later, 10 minutes later, 15 minutes later, 20 minutes later, 30 minutes later, 45 minutes later, 1 hour later, 2 hours later, 3 hours later, 4 hours later, 5 hours later, 6 hours later, 7 hours later, 8 hours later, nine hours later, 10 hours later, 11 hours later, 12 hours later, 13 hours later, 14 hours later, 15 hours later, 16 hours later, 17 hours later, 18 hours later, 19 hours later, 20 hours later, 21 hours later, 22 hours later, 23 hours later, 24 hours later, 25 hours later, 36 hours later, 48 hours later, 76 hours later, 96 hours later, 120 hours later, 144 hours later and 168 hours later, etc.

In another aspect, the methods of the present invention also contemplate that the one or more xanthine oxidoreductase inhibitors and one or more anti-inflammatory agents can be administered (or dosed) as a unified, single pharmaceutical formulation or dosage form. Such formulations can be made using routine techniques known in the art. Additionally, such a formulation can be optionally coated with one or more enteric coatings. For example, a capsule or tablet can be prepared to contain one or more xanthine oxidoreductase inhibitors and one or more anti-inflammatory agents. Alternatively, a solid formulation can be prepared having a core containing one or more xanthine oxidoreductase inhibitors. This core than can be coated with one or more anti-inflammatory agents.

In yet another aspect, the methods of the present invention optionally comprise administering to the subject one or more PPIs. The one or more PPIs can be administered (or dosed) to a subject as a separate independent formulation and thus can be administered sequentially with one or more formulations containing one or more xanthine oxidoreductase inhibitors, one or more formulations containing the one or more anti-inflammatory agents or with a single, unified formulation containing one or more xanthine oxidoreductase inhibitors and one or more anti-inflammatory agents (meaning that each formulation is administered to the subject one right after another). Alternatively, the formulations can be administered to the subject intermittently on the same day or on different days. For example, a formulation containing one or more PPIs can be administered to a subject 5 minutes, 10 minutes, 15 minutes, 20 minutes, 30 minutes, 45 minutes, 1 hour, 2 hours, 3 hours, 4 hours, 5 hours, 6 hours, 7 hours, 8 hours, nine hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 36 hours, 48 hours, 76 hours, 96 hours, 120 hours, 144 hours and 168 hours, etc., after administration of one or more other formulations containing one or more xanthine oxidoreductase inhibitors, after administration of one or more formulations containing one or more anti-inflammatory agents or after administration of a single, unified formulation containing one or more xanthine oxidoreductase inhibitors and one or more anti-inflammatory agents. Additionally, any combination of administrations (dosings) can be used. For example, a subject may be administered a tablet containing one or more anti-inflammatory agents and then immediately administered a tablet containing one or more PPIs. Ten hours later, the subject may be administered a capsule containing one or more xanthine oxidoreductase inhibitors. By way of another example, a subject may be administered a tablet containing one or more xanthine oxidoreductase inhibitors and then 36 hours later be administered a single capsule containing one or more anti-inflammatory agents and one or more PPIs. By way of yet another example, a subject may be administered a tablet containing one or more xanthine oxidoreductase inhibitors followed immediately by a capsule containing one or more anti-inflammatory agents and one or more PPIs.

In yet still another aspect, the one or more PPIs can be administered as a single, unified pharmaceutical formulation along with one or more xanthine oxidoreductase inhibitors, one or more NSAIDs or one or more xanthine oxidoreductase inhibitors and one or more anti-inflammatory agents. Such formulations can be prepared using routine techniques known in the art. Such formulations can also optionally contain one or more enteric coatings. For example, a capsule can be formulated to containing one or more xanthine oxidoreductase inhibitors, one or more anti-inflammatory agents and one or more PPI's. Alternatively, a capsule or tablet can be prepared containing one or more xanthine oxidoreductase inhibitors and one or more PPIs. In still another alternative, a capsule or tablet can be prepared containing one or more anti-inflammatory agents and one or more PPIs. Pharmaceutical formulations containing one or more PPIs and one or more anti-inflammatory agents are well known in the art and are described in U.S. Patent Application Nos. 20020155153; 20040131676; 20040022846; 20050163847; and 2005024984.

The time at which the PPI is administered to a subject is not critical. Preferably, however, the PPI is administered to a subject either before or after the administration of one or more formulations containing one or more anti-inflammatory agents or together with (such as in a single, unified formulation) one or more anti-inflammatory agents. Additionally, the one or more PPIs can be administered to the subject throughout the entire treatment period (namely, the at least six (6) months) or only periodically during the treatment period (such as for 1 day, 2 days, 3 days, 4 days, 5 days, 6 days, 7 days, 14 days, 21 days 1 month, 2 months, 3 months, 4 months 5 months, etc.) when the subject is receiving the one or more xanthine oxidoreductase inhibitors and one or more anti-inflammatory agents.

In yet still another aspect, the methods of the present invention contemplate that the one or more xanthine oxidoreductase inhibitors and one or more anti-inflammatory agents and optionally, one or more PPIs, are administered to the subject on a regular basis. As used herein, the phrase “regular basis” refers to the administration of one or more xanthine oxidoreductase inhibitors, one or more anti-inflammatory agents and, optionally, one or more PPIs, at a time and in an amount that is required to treat the subject, namely, to reduce the number of gout flares experienced by the subject or to prevent the subject from experiencing any gout flares during the treatment period of at least six (6) months. For example, for some subjects, the phrase “regular basis” may mean that during the at least six (6) month period a subject is administered one or more xanthine oxidoreductase inhibitors once or twice a day as well as one or more anti-inflammatory agents once or twice a day. Optionally, the subject may also be administered one or more PPIs once or twice a day during the at least six (6) month treatment period. Alternatively, for other subjects, the phrase “regular basis” may mean that during the at least six (6) month treatment period that the subject is administered one or more xanthine oxidoreductase inhibitors once or twice a day and one or more anti-inflammatory agents once or twice a day every other day or once or twice a day one day a week. Optionally, the subject may also be administered one or more PPIs once or twice every day, once or twice every other day or once or twice one day a week during the at least six (6) month period. In still yet another alternative, for other subjects, the phrase “regular basis” may mean that during the six (6) month period, the subject is administered one or more xanthine oxidoreductase inhibitors once or twice a day every other day or once or twice a day once a week and one or more anti-inflammatory agents once or twice a day every day. Optionally, the subject may also be administered one or more PPIs once or twice a day every day during the at least six (6) month period.

Compositions containing at least one xanthine oxidoreductase inhibitor, at least one anti-inflammatory agent and optionally, at least one PPI are contemplated for use in the methods of the present invention. Using the excipients and dosage forms described below, formulations containing such compositions are a matter of choice for those skilled in the art. Further, those skilled in the art will recognize that various coatings or other separation techniques may be used in cases where the combination of compounds are incompatible.

Compounds for use in accordance with the methods of the present invention can be provided in the form of pharmaceutically acceptable salts derived from inorganic or organic acids. Pharmaceutically acceptable salts are well-known in the art. For example, S. M. Berge et al. describe pharmaceutically acceptable salts in detail in J. Pharmaceutical Sciences, 66: 1 et seq. (1977). The salts can be prepared in situ during the final isolation and purification of the compounds or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

The at least one xanthine oxidoreductase inhibiting compound or salts thereof, the at least one anti-inflammatory agents and optionally, at least one PPI may be formulated in a variety of ways that is largely a matter of choice depending upon the delivery route desired. For example, solid dosage forms for oral administration include capsules, tablets, pills, powders and granules. In such solid dosage forms, the at least one xanthine oxidoreductase inhibiting compound, at least one anti-inflammatory agents, at least one PPI or any combinations thereof may be mixed with at least one inert, pharmaceutically acceptable excipient or carrier, such as sodium citrate or dicalcium phosphate and/or a) fillers or extenders, such as, but not limited to, starches, lactose, sucrose, glucose, mannitol and silicic acid; b) binders, such as, but not limited to, carboxymethylcellulose, alginates, gelatin, polyvinylpyrrolidone, sucrose and acacia; c) humectants, such as, but not limited to glycerol; d) disintegrating agents, such as, but not limited to, agar-agar, calcium carbonate, potato or tapioca starch, alginic acid, certain silicates and sodium carbonate; e) solution retarding agents, such as, but not limited to, paraffin; f) absorption accelerators, such as, but not limited to, quaternary ammonium compounds; g) wetting agents, such as, but not limited to, cetyl alcohol and glycerol monostearate; h) absorbents, such as, but not limited to, kaolin and bentonite clay; and i) lubricants, such as, but not limited to, talc, calcium stearate, magnesium stearate, solid polyethylene glycols, sodium lauryl sulfate and mixtures thereof.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

Liquid dosage forms for oral administration include pharmaceutically acceptable emulsions, solutions, suspensions, syrups and elixirs. In addition to the xanthine oxidoreductase inhibiting compounds, one or more anti-inflammatory agents, one or more PPIs or any combinations thereof, the liquid dosage forms may contain inert diluents commonly used in the art such as, for example, water or other solvents, solubilizing agents and emulsifiers, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

The compositions can also be delivered through a catheter for local delivery at a target site, via an intracoronary stent (a tubular device composed of a fine wire mesh), or via a biodegradable polymer.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include, but are not limited to, water, ethanol, polyols (propylene glycol, polyethylene glycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form can be brought about by the use of agents delaying absorption, for example, aluminum monostearate and gelatin.

Suspensions, in addition to the active compounds (i.e., at least one xanthine oxidoreductase inhibiting compounds or salts thereof, at least one anti-inflammatory agent, optionally, at least one PPI and any combinations thereof), may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Proper fluidity can be maintained, for example, by the use of coating materials such as lecithin, by the maintenance of the required particle size in the case of dispersions and by the use of surfactants.

In some cases, in order to prolong the effect of the drug (i.e. xanthine oxidoreductase inhibiting compounds or salts thereof, one or more anti-inflammatory agents, one or more PPIs or any combinations thereof), it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Dosage forms for topical administration of the compounds of this present invention include powders, sprays, ointments and inhalants. The active compound(s) is mixed under sterile conditions with a pharmaceutically acceptable carrier and any needed preservatives, buffers or propellants which can be required. Opthalmic formulations, eye ointments, powders and solutions are also contemplated as being within the scope of this invention.

It will be understood that formulations used in accordance with the present invention generally will comprise a therapeutically effective amount of one or more xanthine oxidoreductase inhibiting compounds, one or more anti-inflammatory agents, one or more PPIs or any combinations thereof.

Formulations of the present invention are administered and dosed in accordance with sound medical practice, taking into account the clinical condition of the individual patient, the site and method of administration, scheduling of administration, and other factors known to medical practitioners.

Therapeutically effective or prophylactically effective amounts of one or more xanthine oxidoreductase inhibitors, one or more anti-inflammatory agents and optionally, one or more PPIs for purposes of the methods described herein thus can readily be determined by such considerations as are known to those skilled in the art (such as in accordance with the appropriate labels, the Physicians Desk Reference, the U.S. Pharmacopeia (“USP”), etc.). The daily therapeutically effective or prophylactically effective amount of the xanthine oxidoreductase inhibiting compounds administered to a patient in single or divided doses range from about 0.01 to about 750 milligram per kilogram of body weight per day (mg/kg/day). More specifically, a patient may be administered from about 5.0 mg to about 300 mg once daily, preferably from about 20 mg to about 240 mg once daily and most preferably from about 40 mg to about 80 mg once daily of xanthine oxidoreductase inhibiting compounds. It is preferred the patient be administered from about 40 mg to about 80 mg daily of febuxostat, about 250 mg to about 1000 mg daily of naproxen and optionally, at least 15 mg to about 30 mg daily of lansoprazole. Of course, it will be understood by one skilled in the art that other dosage regimens may be utilized, such as dosing more than once per day or more than twice a day, utilizing extended, controlled, or modified release dosage forms, and the like in order to achieve the desired result of reducing or preventing the number of gout flares experienced by a patient during the at least six (6) month period.

The present invention also includes a pharmaceutical kit, preferably an oral pharmaceutical kit. The pharmaceutical kit of the present invention comprises as active ingredients a therapeutically effective amount of: (1) at least one xanthine oxidoreductase inhibitor; and (2) at least one anti-inflammatory agents. Optionally, the kit can also further comprise a therapeutically effective amount of at least one PPI. In the kit of the present invention, the at least one xanthine oxidoreductase inhibitor and the at least one anti-inflammatory agent can each be provided as separate, independent dosage forms (namely, as at least two dosage forms, such as two tablets, two capsules, a tablet and a capsule, etc.). Alternatively, the at least one xanthine oxidoreductase inhibitor and the at least one anti-inflammatory agent can be combined in a single, unified dosage form (such as a single tablet or a single capsule). In still another alternative, the at least one xanthine oxidoreductase inhibitor, the at least one anti-inflammatory agent and at least one PPI can each be provided as separate, independent dosage forms (namely, as at least three dosage forms, such as three tablets or three capsules, one tablet and two capsules, two tablets and one capsule, etc). In yet still another alternative, the at least one xanthine oxidoreductase inhibitor, the at least one anti-inflammatory agent and at least one PPI can be combined in a single, unified dosage form (such as a single tablet or a single capsule). In yet still a further alternative, the at least one xanthine oxidoreductase inhibitor and at least one PPI can be combined in a single, unified dosage form (such as a single tablet or a single capsule) and the at least one anti-inflammatory agent can be provided as a separate, independent dosage form (such as a single tablet or a single capsule). In still another alternative, the at least one anti-inflammatory agent the and at least one PPI can be combined in a single, unified dosage form (such as a single tablet or a single capsule) and the at least one xanthine oxidoreductase inhibitor can be provided as a separate, independent dosage form (such as a single tablet or a single capsule). The kit of the present invention can be used in the methods described herein. For example, the kit can be used to prevent gout flares in a subject in need of treatment thereof for a period of at least six (6) months or to reduce the number of gout flares in a subject in need of treatment thereof for a period of at least six (6) months.

By way of example, and not of limitation, examples of the present invention will now be given.

### EXAMPLE 1

This example describes a Phase 3 study that is designed to evaluate the efficacy and safety of febuxostat 40 mg administered once a day (“QD”) versus allopurinol in lowering serum urate in subjects with hyperuricemia associated with gout. Febuxostat 80 mg QD is also included in this study as a reference treatment group. Renal impairment is frequently observed in subjects with gout. Therefore, subjects who have renal impairment will also be included in this study.

Study Design:

This will be a Phase 3, randomized, double-blind, multicenter, active-controlled study with a 6-month treatment period.

Patient Population:

Approximately 2,250 subjects with a serum urate (“sUA”) level ≧8.0 mg/dL, with a history or presence of gout based on American Rheumatology Association (“ARA”) criteria, will be enrolled at approximately 300 sites in the U.S.

### Treatments:

Subjects will undergo a 30-day Washout Period (Day-30 Screening Visit), if currently taking urate-lowering therapy (“ULTs”); no Washout Period is required for subjects not on prior ULTs.
Subjects will be randomized in a 1:1:1 ratio to one of three treatment groups:
   Febuxostat 40 mg QD
   Febuxostat 80 mg QD
   Allopurinol [200 mg QD if renal impairment (defined as estimated creatinine clearance ≧20 and <80 mL/min) or 300 mg QD if normal renal function (ie, estimated creatinine clearance ≧80 mL/min)].
Randomization will be stratified, based on renal function.

Gout Flare Prophylaxis:

Gout flare prophylaxis, consisting of 0.6 mg QD colchicine will be provided for the duration of the study. Alternatively, if colchicine is not tolerated by the subject, and the subject's creatinine clearance is ≧50 ml/min he/she will be administered naproxen 250 mg BID with lansoprazole 15 mg QD. Subjects with a creatinine clearance of <50 ml/min generally should not receive naproxen. Alternate treatment options will be provide for such subjects with a creatinine clearance of <50 ml/min.

For those subjects who were on urate-lowering therapy prior to the start of the study, gout flare prophylaxis will be administered at the Day-30 Screening visit (during the 30-day washout period) and for the duration of the study. A washout period will not be required for a subject not on prior ULTs.

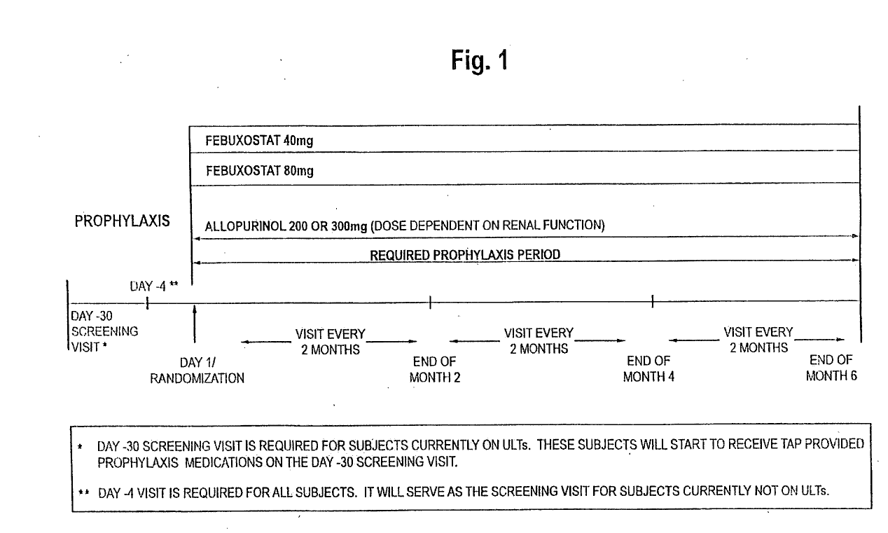

FIG. 1 provides a detailed schematic of the study design.

Inclusion Criteria:
Subjects will be male or female and between the ages of 18 to 85 years;
Subject is defined as having one or more of the ARA criteria for the diagnosis of gout;
Female subjects must be:
   Postmenopausal (defined as amenorrhea for at least 2 years and age ≧50 years), or
   Surgically sterile (including bilateral tubal ligation and/or hysterectomy), or
   Using a medically accepted means of contraception and have a negative pregnancy test prior to enrollment. Medically accepted means of contraception are oral or injectable hormonal contraceptives or intrauterine systems with progestin used for >90 days prior to Day 1, throughout the study and for 30 days after the last dose or barrier method contraceptives (condom with spermicides or intrauterine device) used during the Screening Period, throughout the study and for 30 days after the last dose, or continuous practice of abstinence (when abstinence is discontinued during this period, a barrier contraception must be used).
Subject must have a sUA level ≧8.0 mg/dL at the Day-4 Visit

Exclusion Criteria:
Subject has a history of xanthinuria;
Subject has received urate-lowering therapy (ie, allopurinol, probenecid, etc.) other than the study drug;
Long term use of NSAIDs and COX-2 inhibitors, salicylates; thiazide diuretics; losartan; azathioprine; mercaptopurine; theophylline; IV colchicine; cyclosporine; cyclophosphamide; pyrazinamide; sulfamethoxazole; trimethoprim.
Subject has a known hypersensitivity to febuxostat or allopurinol or any components of their formulation; subject has a known hypersensitivity to naproxen, any other NSAID, aspirin, lansoprazole, or colchicine, or any components in their formulation;
Subject has rheumatoid arthritis which requires treatment;
Subject has a severe, unstable or life threatening medical condition that would likely prevent them from completing this study;
Subject consumes >14 alcoholic beverages/week.
Active liver disease or peptic ulcer disease.
History of significant concomitant illness.
Subject's estimated creatinine clearance is <30 mL/min, where creatinine clearance is calculated using the Cockcroft and Gault formula for Ideal Body Weight, as provided below:

$$Estimated creatinine clearance = \frac{( 140 - age [ yr ] ) \times ( IBW [ kg ] )}{72 \times ( Scr [ mg / dL ] )} ( females multiply by 0.85 )$$

Where IBW (ideal body weight) is 50 kg for males and 45.5 kg for women, plus 2.3 kg for each inch in height >5 feet (60 inches).

Efficacy

Primary Efficacy Endpoint:

The primary efficacy endpoint will be the proportion of subjects whose sUA level is <6.0 mg/dL at the Final Visit.

Secondary Efficacy Endpoints:
1. The proportion of renal impairment subjects whose Final Visit serum urate level is <6.0 mg/dL.
2. The proportion of subjects whose serum urate levels are <6.0 mg/dL, <5.0 mg/dL and <4.0 mg/dL, at each visit.
3. The percent reduction from baseline in serum urate levels, at each visit.

While the invention has been described by reference to certain presently preferred embodiments, it will be understood that modifications and variations thereof apparent to those skilled in the art are intended to be included within the scope of the invention.

What is claimed is:

1. A method of preventing one or more gout flares in a subject in need thereof, the method comprising the step of:
administering to the subject on a regular basis and for a period of at least six months, a therapeutically effective amount of at least one xanthine oxidoreductase inhibitor or a pharmaceutically acceptable salt thereof and a therapeutically effective amount of at least one anti-inflammatory agent.

2. The method of claim 1, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo[1,5-a]-1,3,5-triazin-4-(1H)-one, 8-[3-methoxy-4-(phenylsulfinyl)phenyl]-sodium salt (O), 3-(2-methyl-4-pyridyl)-5-cyano-4-isobutoxyphenyl)-1,2,4-triazole and a pharmaceutically acceptable salt thereof.

3. The method of claim 1, wherein the subject has hyperuricemia, gout, acute gouty arthritis, chronic gouty joint disease, tophaceous gout, uric acid nephropathy, or nephrolithiasis.

4. The method of claim 1, wherein the at least one anti-inflammatory agent is colchicine or a non-steroidal anti-inflammatory agent (“NSAID”).

5. The method of claim 4, wherein the NSAID is selected from the group consisting of: acetaminophen, amoxiprin, benorilate, choline magnesium salicylate, difunisal, faislamine, methyl salicylate, magnesium salicylate, salicyl salicylate, diclofenac, aceclofenac, acemetacin, bromfenac, etodolac, ketorolac, nabumetone, sulindac, tolmetin, ibuprofen, carprofen, fenbufen, fenoprofen, flurbiprofen, ketoprofen, loxoprofen, naproxen, tiaprofenic acid, mefenamic acid, meclofenamic acid, tolfenamic acid, phenylbutazone, azapropazone, metamizole, oxyphenbutazone, piroxicam, lomoxicam, meloxicam, tenoxicam, celecoxib, etoricoxib, lumiracoxib, parecoxib, nimesulide, licofelone, indomethacin, a COX-2 inhibitor and pharmaceutically acceptable salts thereof and mixtures thereof.

6. The method of claim 5, wherein the NSAID is naproxen.

7. The method of claim 1, further comprising administering to the subject a therapeutically effective amount of at least one proton pump inhibitor (“PPI”).

8. The method of claim 7, wherein the PPI is lansoprazole, ilaprazole, omeprazole, tenatoprazole, rabeprazole, esomeprazole, pantoprazole, pariprazole, leminoprazole or nepaprazole or a free base, a free acid, a salt, a hydrate, an ester, an amide, an enantiomer, an isomer, a tautomer, a polymorph, a prodrug or any derivative thereof.

9. The method of claim 8, wherein the PPI is lansoprazole.

10. A method of preventing one or more gout flares in a subject in need thereof, the method comprising the step of:
administering to the subject on a regular basis and for a period of at least six months, a therapeutically effective amount of at least one non-steroidal anti-inflammatory drug (“NSAID”) and a therapeutically effective amount of a second compound or a pharmaceutically acceptable salt thereof, wherein said second compound comprises the formula: 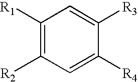
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R₄ are each independently a hydrogen or A, B, C or D as shown below: 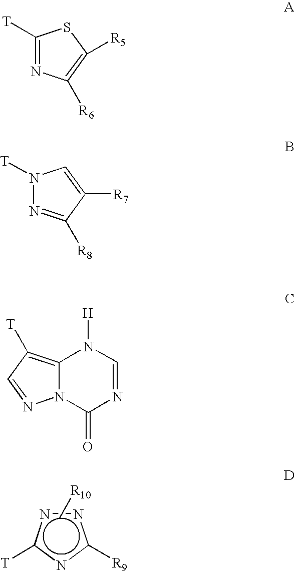
wherein T connects A, B, C or D to the aromatic ring shown above at R₁, R₂, R₃ or R₄.
wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

11. The method of claim 10, wherein the second compound is 2-[3-cyano-4-(2-methylpropoxy)phenyl]-4-methylthiazole-5-carboxylic acid or a pharmaceutically acceptable salt thereof.

12. The method of claim 10, wherein the second compound is 2-[3-cyano-4-(3-hydroxy-2-methylpropoxy)phenyl]-4-methyl-5-thiazolecarboxylic acid or a pharmaceutically acceptable salt thereof.

13. The method of claim 10, wherein the second compound is 2-[3-cyano-4-(2-hydroxy-2-methylpropoxy)phenyl]-4-methyl-5-thiazolecarboxylic acid or a pharmaceutically acceptable salt thereof.

14. The method of claim 10, wherein the second compound is 2-(3-cyano-4-hydroxyphenyl)-4-methyl-5-thiazolecarboxylic acid or a pharmaceutically acceptable salt thereof.

15. The method of claim 10, wherein the second compound is 2-[4-(2-carboxypropoxy)-3-cyanophenyl]-4-methyl-5-thiazolecarboxylic acid or a pharmaceutically acceptable salt thereof.

16. The method of claim 10, wherein the second compound is 1-3-cyano-4-(2,2-dimethylpropoxy)phenyl]-1H-pyrazole-4-carboxylic acid or a pharmaceutically acceptable salt thereof.

17. The method of claim 10, wherein the second compound is pyrazolo[1,5-a]-1,3,5-triazin-4-(1H)-one, 8-[3-methoxy-4-(phenylsulfinyl)phenyl]-sodium salt (O).

18. The method of claim 10, wherein the second compound is 3-(2-methyl-4-pyridyl)-5-cyano-4-isobutoxyphenyl)-1,2,4-triazole or a pharmaceutically acceptable salt thereof.

19. The method of claim 10, wherein the subject has hyperuricemia, gout, acute gouty arthritis, chronic gouty joint disease, tophaceous gout, uric acid nephropathy, or nephtolithiasis.

20. The method of claim 10, wherein the at least one anti-inflammatory agent is colchicine or a non-steroidal anti-inflammatory agent (“NSAID”).

21. The method of claim 20, wherein the NSAID is selected from the group consisting of: acetaminophen, amoxiprin, benorilate, choline magnesium salicylate, difunisal, faislamine, methyl salicylate, magnesium salicylate, salicyl salicylate, diclofenac, aceclofenac, acemetacin, bromfenac, etodolac, ketorolac, nabumetone, sulindac, tolmetin, ibuprofen, carprofen, fenbufen, fenoprofen, flurbiprofen, ketoprofen, loxoprofen, naproxen, tiaprofenic acid, mefenamic acid, meclofenamic acid, tolfenamic acid, phenylbutazone, azapropazone, metamizole, oxyphenbutazone, piroxicam, lomoxicam, meloxicam, tenoxicam, celecoxib, etoricoxib, lumiracoxib, parecoxib, nimesulide, licofelone, indomethacin, a COX-2 inhibitor and pharmaceutically acceptable salts thereof and mixtures thereof.

22. The method of claim 21, wherein the NSAID is naproxen.

23. The method of claim 10, further comprising the step of administering to the subject a therapeutically effective amount of at least one proton pump inhibitor (“PPI”).

24. The method of claim 23, wherein the PPI is lansoprazole, ilaprazole, omeprazole, tenatoprazole, rabeprazole, esomeprazole, pantoprazole, pariprazole, leminoprazole or nepaprazole or a free base, a free acid, a salt, a hydrate, an ester, an amide, an enantiomer, an isomer, a tautomer, a polymorph, a prodrug or any derivative thereof.

25. The method of claim 24, wherein the PPI is lansoprazole.

26. A method of preventing one or more gout flares in a subject in need thereof, the method comprising the step of:
administering to the subject on a regular basis and for a period of at least six months, a therapeutically effective amount of at least one non-steroidal anti-inflammatory drug (“NSAID”) and a therapeutically effective amount of a second compound or a pharmaceutically acceptable salt thereof, wherein said second compound comprises the formula: 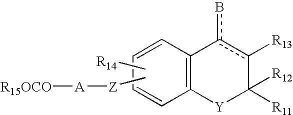
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or a ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds.

27. The method of claim 26, wherein the subject has hyperuricemia, gout, acute gouty arthritis, chronic gouty joint disease, tophaceous gout, uric acid nephropathy, or nephtolithiasis.

28. The method of claim 26, wherein the at least one anti-inflammatory agent is colchicine or a non-steroidal anti-inflammatory agent (“NSAID”).

29. The method of claim 28, wherein the NSAID is selected from the group consisting of: acetaminophen, amoxiprin, benorilate, choline magnesium salicylate, difunisal, faislamine, methyl salicylate, magnesium salicylate, salicyl salicylate, diclofenac, aceclofenac, acemetacin, bromfenac, etodolac, ketorolac, nabumetone, sulindac, tolmetin, ibuprofen, carprofen, fenbufen, fenoprofen, flurbiprofen, ketoprofen, loxoprofen, naproxen, tiaprofenic acid, mefenamic acid, meclofenamic acid, tolfenamic acid, phenylbutazone, azapropazone, metamizole, oxyphenbutazone, piroxicam, lomoxicam, meloxicam, tenoxicam, celecoxib, etoricoxib, lumiracoxib, parecoxib, nimesulide, licofelone, indomethacin, a COX-2 inhibitor and pharmaceutically acceptable salts thereof and mixtures thereof.

30. The method of claim 29, wherein the NSAID is naproxen.

31. The method of claim 26, further comprising the step of administering to the subject a therapeutically effective amount of at least one proton pump inhibitor (“PPI”).

32. The method of claim 31, wherein the PPI is lansoprazole, ilaprazole, omeprazole, tenatoprazole, rabeprazole, esomeprazole, pantoprazole, pariprazole, leminoprazole or nepaprazole or a free base, a free acid, a salt, a hydrate, an ester, an amide, an enantiomer, an isomer, a tautomer, a polymorph, a prodrug or any derivative thereof.

33. The method of claim 32, wherein the PPI is lansoprazole.

34. A pharmaceutical kit comprising as active ingredients a therapeutically effective amount of: (1) at least one xanthine oxidoreductase inhibitor; and (2) at least one anti-inflammatory agent.

35. The kit of claim 34, wherein the kit further comprises a therapeutically effective amount of at least one proton pump inhibitor (“PPI”).

36. The kit of claim 34, wherein the at least one xanthine oxidoreductase inhibitor and the at least one anti-inflammatory agent are each provided as separate, independent dosage forms.

37. The kit of claim 34, wherein the at least one xanthine oxidoreductase inhibitor and the at least one anti-inflammatory agent are combined in a single, unified dosage form.

38. The kit of claim 35, wherein the at least one xanthine oxidoreductase inhibitor, the at least one anti-inflammatory agent and at least one PPI are each provided as separate, independent dosage forms.

39. The kit of claim 35, wherein the at least one xanthine oxidoreductase inhibitor, the at least one anti-inflammatory agent and the at least one PPI are combined in a single, unified dosage form.

40. The kit of claim 35, wherein the at least one xanthine oxidoreductase inhibitor and at least one PPI are combined in a single, unified dosage form and the at least one anti-inflammatory agent is provided as a separate, independent dosage form.

41. The kit of claim 35, wherein the at least one anti-inflammatory agent and the at least one PPI are combined in a single, unified dosage form and the at least one xanthine oxidoreductase inhibitor is provided as a separate, independent dosage form.

42. The kit of claim 34, wherein the at least one anti-inflammatory agent is colchicine or an NSAID.

43. The kit of claim 42, wherein the NSAID is selected from the group consisting of: acetaminophen, amoxiprin, benorilate, choline magnesium salicylate, difunisal, faislamine, methyl salicylate, magnesium salicylate, salicyl salicylate, diclofenac, aceclofenac, acemetacin, bromfenac, etodolac, ketorolac, nabumetone, sulindac, tolmetin, ibuprofen, carprofen, fenbufen, fenoprofen, flurbiprofen, ketoprofen, loxoprofen, naproxen, tiaprofenic acid, mefenamic acid, meclofenamic acid, tolfenamic acid, phenylbutazone, azapropazone, metamizole, oxyphenbutazone, piroxicam, lomoxicam, meloxicam, tenoxicam, celecoxib, etoricoxib, lumiracoxib, parecoxib, nimesulide, licofelone, indomethacin, a COX-2 inhibitor and pharmaceutically acceptable salts thereof and mixtures thereof.

44. The kit of claim 43, wherein the NSAID is naproxen.

45. The kit of claim 35, wherein the PPI is lansoprazole, ilaprazole, omeprazole, tenatoprazole, rabeprazole, esomeprazole, pantoprazole, pariprazole, leminoprazole or nepaprazole or a free base, a free acid, a salt, a hydrate, an ester, an amide, an enantiomer, an isomer, a tautomer, a polymorph, a prodrug or any derivative thereof.

46. The kit of claim 45, wherein the PPI is lansoprazole.
